(19) United States
(12) Patent Application Publication
(10) Pub. No.: US 2024/0299193 A1
(43) Pub. Date: Sep. 12, 2024

(54) PROSTHETIC HAND WITH WATERPROOF COVER AND FILAMENT ATTACHMENT FOR DIGITS

(71) Applicant: Touch Bionics Limited, Livingston (GB)

(72) Inventors: Rodrigo Mercader Rivera, Livingston (GB); Matthew James Benning, Livingston (GB); Abram David Yeudall, Livingston (GB); Gabriele Cattelan, Livingston (GB); Stephen Pemberton, Livingston (GB); Alexander Campbell, Glasgow (GB)

(21) Appl. No.: 18/596,427

(22) Filed: Mar. 5, 2024

Related U.S. Application Data
(60) Provisional application No. 63/488,874, filed on Mar. 7, 2023.

(51) Int. Cl. A61F 2/58; A61F 2/50

(52) U.S. Cl. CPC A61F 2/586; A61F 2002/5089; A61F 2002/587

(57) ABSTRACT

A prosthetic hand with a waterproof cover and/or a filament-based digit securement mechanism. A first annular recess on the digit aligns with a second corresponding annular recess on a chassis to form a groove into which the filament is inserted. The prosthetic hand may comprise a waterproof cover that protects the interior components of the prosthetic hand. A thumb cover and a dorsal cover attach to a palm cover. A thumb cover interface is adhered via a clamp with an exterior sealing ring. A dorsal cover interface has a sealing ring. The thumb cover may be flexible and have three layers. The dorsal cover may be rigid and have a latch that snaps onto the chassis. The cover may be put onto the palm portion with the thumb and thumb cover, and then the prosthetic digits may be attached to the chassis thereafter.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Patent Application No. 63/488,874, titled “PROSTHETIC HAND WITH WATERPROOF COVER AND FILAMENT ATTACHMENT FOR DIGITS” and filed Mar. 7, 2023, which is incorporated herein by reference in its entirety for all purposes and forms a part of this specification.

### BACKGROUND

### Field

This disclosure relates to prosthetic hands, in particular to waterproof covers for prosthetic hands and filament attachments for attaching prosthetic digits to a palm portion.

### Description of the Related Art

Prosthetics are used to replace amputated natural body parts. Prosthetic hands, which can include prosthetic digits and a thumb, may be used to replace amputated natural hands. Attachment of prosthetic digits to prosthetic palm portions is important for assembly and for when digits need replacing. Conventional attachment of prosthetic digits to the palm portion require screws, which require high tolerancing and are prone to loosening. Existing solutions also use multiple screws and are complex to change the digit, require longer assembly times, create difficulty with assembling small threaded fasteners, and only have small fixation surface areas. Further, such approaches require additional tools to remove and fasten the screws. Existing solutions for prosthetic hands thus require complex removal and replacement procedures for individual prosthetic digits.

Additionally, prosthetic hand covers are important for providing an outer surface that mimics that of a natural hand to aid in various movements while protecting interior components from water and other fluid elements. Conventional prosthetic hand cover solutions require screws to attach the cover and assembly methods that first require attachment of prosthetic digits prior to attaching the cover, or conversely first require removal of the cover prior to removal or replacement of the prosthetic digits. Such solutions also are not waterproof.

Improvements to these and other drawbacks of existing solutions for prosthetic hands are desirable.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for prosthetic digit attachment and prosthetic hand covers.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In one aspect, a prosthetic hand comprises a palm portion, a prosthetic digit and a prosthetic thumb extending from the palm portion, a chassis, a flexible filament, and a cover assembly. The chassis is located within the palm portion and attached to the prosthetic digit to define an annular groove extending between the prosthetic digit and the chassis. The flexible filament extends through the groove. The cover assembly comprises a palm cover, a thumb cover, and a dorsal cover. The palm cover extends partially over the palm portion and defines a lateral opening and a dorsal opening. The thumb cover extends around at least part of the prosthetic thumb and is secured to the lateral opening of the palm cover. The dorsal cover extends over the dorsal opening.

There are various embodiments of the various aspects. The above and other aspects may include the following: the prosthetic digit further comprises a first recess extending annularly about a proximal end of the prosthetic digit and that partially defines the annular groove; the chassis further comprises an opening at a distal end of the chassis, the opening having a second recess extending annularly around an inner surface of the opening, where the opening is configured to receive therein the proximal end of the prosthetic digit, such that the first recess and the second recess align to define the annular groove between the prosthetic digit and the chassis; a thumb clamp, the thumb clamp adhered to the thumb cover and the lateral opening along a thumb interface, with an exterior thumb seal extending over an exterior of the thumb interface, such that the thumb interface is impermeable to liquid; the dorsal cover is rigid and comprises an outer perimeter extending at least partially along a corresponding inner perimeter of the dorsal opening along a dorsal interface, and the dorsal cover is secured to the chassis via a latch, such that the dorsal interface is impermeable to liquid; the thumb cover is flexible and comprises an internal elastane layer, a middle rubberized layer, and a woven fabric external layer; and/or four of the prosthetic digits and four of the filaments, with each filament extending through a respective groove defined by the respective prosthetic digit and the chassis.

In another aspect, the prosthetic hand comprises a prosthetic digit, a chassis, and a flexible filament. The prosthetic digit comprises a first recess extending annularly about an outer surface of a proximal end of the prosthetic digit. The chassis is carried by a palm portion of the prosthetic hand, the chassis having a distal end comprising an opening having a second recess extending annularly around an inner surface of the opening, where the opening is configured to receive therein the proximal end of the prosthetic digit such that the first recess and the second recess align to define a groove between the prosthetic digit and the chassis. The flexible filament extends through the groove and thereby secures the prosthetic digit to the chassis.

There are various embodiments of the various aspects. The above aspects and other aspects may include the following: the chassis further comprises an access opening leading to the annular groove, the access opening configured to receive a distal end of the filament therein for insertion of the filament into the groove; a cover extending around the palm portion, and where a proximal end of the filament is accessible to a user via the access opening; the access opening is wider than the groove, and where, with the filament fully inserted into the groove, a proximal end of the filament comprises an end portion configured to be received within the access opening; the first recess and the second recess each have cross sections with a long side and two short sides perpendicular to the long side such that, with the first recess and the second recess aligned, the groove defines a four-sided cross section; the groove comprises a proximal end extending to a distal end, and where the distal end is open to the proximal end to define a continuous annular pathway; and/or the filament comprises a longitudinal core with an outer strand wrapped around the core.

In another aspect, a prosthetic hand comprises a palm portion, a prosthetic digit, a prosthetic thumb, and a cover assembly. The palm portion has a distal end, a palm side opposite a dorsal side, and a lateral inner side opposite a lateral outer side. The prosthetic digit is attached to and extending from the distal end of the palm portion. The prosthetic thumb is attached to and extending from the lateral inner side of the palm portion. The cover assembly comprises a polymeric palm cover, a ridge of the palm cover, a palm sealing ring, a flexible thumb cover, a thumb clamp, an exterior thumb seal, and a rigid dorsal cover. The polymeric palm cover extends partially around the palm portion and defines a lateral opening on the lateral inner side of the palm portion, a distal opening on the distal end of the palm portion, and a dorsal opening on the dorsal side of the palm portion. The ridge of the palm cover is located proximally of the distal opening and protrudes inwardly from and extends at least partially along one or more inner surfaces of the palm cover. The palm sealing ring extends along the ridge of the palm cover with a chassis compressing the palm sealing ring against the ridge along a palm interface, where the palm interface is impermeable to liquid. The flexible thumb cover has a sidewall with a closed cross-section and extends from a proximal thumb opening to a distal thumb opening, the proximal thumb opening attached to the lateral opening of the palm cover and a distal end of the prosthetic thumb extending distally from the distal thumb opening. The thumb clamp extends along and secures a first perimeter of the lateral opening of the palm cover with a second perimeter of the proximal thumb opening of the thumb cover along a thumb cover interface. The exterior thumb seal extends over an exterior of the thumb cover interface, where the thumb cover interface is impermeable to liquid. The rigid dorsal cover has an outer perimeter extending at least partially along a corresponding inner perimeter of the dorsal opening along a dorsal interface, where the dorsal interface is impermeable to liquid.

There are various embodiments of the various aspects. The above aspects and other aspects may include the following: the exterior thumb seal comprises a deformable grip pad facing outwardly from the palm portion; the thumb cover comprises an internal elastane layer, a middle rubberized layer, and a woven fabric external layer; a chassis within the palm portion, where the prosthetic digit and the chassis define an annular groove, and a filament located within the groove thereby securing the prosthetic digit to the chassis; a dorsal opening defined by the chassis, and a latch attached to and extending away from an interior of the dorsal cover, the latch secured within the dorsal opening of the chassis; and/or a dorsal sealing ring extending along the inner perimeter of the dorsal opening.

In another aspect, a method of attaching a prosthetic digit to a prosthetic hand comprises placing a proximal end of a prosthetic digit within an opening of a chassis, forming a groove along an interface of the prosthetic digit and the chassis, and inserting a filament via an access opening of the chassis into the groove, thereby securing the prosthetic digit with the chassis.

In another aspect, a method of attaching a cover to a prosthetic hand comprises inserting a chassis into a palm cover through a distal opening of the palm cover, and attaching, after the chassis has been inserted into the palm cover, a prosthetic digit to a distal end of the chassis.

There are various embodiments of the various aspects. The above aspects and other aspects may include the following: attaching, prior to inserting the chassis into the palm cover, a thumb cover about a prosthetic thumb that is attached to the chassis; and/or attaching a dorsal cover to a dorsal side of the palm cover.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a dorsal view of an example of a prosthetic hand having four prosthetic digits attached via filaments, a prosthetic thumb, and a waterproof cover.

FIG. 2 is perspective view showing a chassis from the prosthetic hand of FIG. 1, with partial prosthetic digits attached to the chassis.

FIGS. 3A and 3B are perspective views of the chassis and prosthetic hand of FIG. 1 respectively showing a filament being inserted into an access opening of a groove on a palm side of the chassis to secure the prosthetic digit with the chassis.

FIGS. 4A-4C are various cross-section views taken from FIGS. 3A and 3B showing the filament within the chassis groove.

FIGS. 5A and 5B are a partial palm-side view and a partial cross-section view respectively of the prosthetic hand of FIG. 1 showing example access openings that lead to the groove.

FIG. 6 is a perspective view showing another example embodiment of a chassis that may be used with the prosthetic hand of FIG. 1 having access openings for filaments on a dorsal side of the chassis.

FIG. 7 is a close up view of a digit opening of the chassis of FIG. 6 showing first and second grooves.

FIGS. 8A and 8B are closeup partial cross-section views taken across line A-A in FIG. 1 showing an example of the filament and the digit seal.

FIG. 9 is a perspective, partial cross-section view of an example filament having a longitudinal core and outer winding and that may be used with the prosthetic hand of FIG. 1.

FIG. 10 is a flow chart illustrating an example method of attaching a prosthetic digit to a chassis.

FIGS. 11A and 11B are perspective dorsal side and palm side views, respectively, of the prosthetic hand of FIG. 1, with an example waterproof cover having a palm cover attached to a thumb cover and a dorsal cover.

FIG. 12A is an exploded view of a palm portion of the prosthetic hand of FIG. 1, showing the palm cover, a thumb clamp, and the thumb cover.

FIG. 12B is a perspective view of the thumb cover of FIG. 12A showing a three-layer thumb cover.

FIG. 13 is a detailed palm side perspective view of an embodiment of a grip pad and thumb tip that may be included with the cover of FIGS. 11A and 11B.

FIG. 14 is a cross-section view of the grip pad and thumb tip of FIG. 10.

FIGS. 15A and 15B are detailed perspective views of a detachable thumb grip attachment that may be included with the cover of FIGS. 8A and 8B.

FIG. 16 is an exploded view of part of the prosthetic hand of FIG. 1, showing of an example assembly method of the palm cover, the chassis, and a palm sealing ring.

FIG. 17 is a partial cross-section view of the assembly of FIG. 13, showing the assembled arrangement of the palm sealing ring sandwiched between a ridge of the palm cover and the chassis.

FIGS. 18A-18C are various perspective views of the palm portion and dorsal cover of the prosthetic hand of FIG. 1, showing the dorsal sealing ring on the palm portion and a latch on the dorsal cover.

FIG. 19 is a flow chart illustrating an example method of assembling the waterproof cover and palm portion of the prosthetic hand.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

The present disclosure is directed to a prosthetic hand containing a filament-based digit securement mechanism and a waterproof cover. The prosthetic hand may include one or both of the filament-based digit securement mechanism and the waterproof cover. The securement mechanism is used to attach the four “non-thumb” digits to a palm portion, although it may be used for attaching a prosthetic thumb as well. The cover provides a waterproof protective element to the palm portion of the prosthetic hand. A first annular recess on the digit aligns with a second corresponding annular recess on a chassis to form a groove into which the filament is inserted. The waterproof cover protects the interior components of the prosthetic hand. A thumb cover and a dorsal cover attach to a palm cover. A thumb cover interface is adhered via a clamp with an exterior sealing ring. A dorsal cover interface has a sealing ring. The thumb cover may be flexible and have three layers. The dorsal cover may be rigid and have a latch that snaps onto the chassis. The cover may be put onto the palm portion with the thumb and thumb cover, and then the prosthetic digits may be attached to the chassis thereafter. These and other features will now be described in detail.

### A. Example Prosthetic Hand

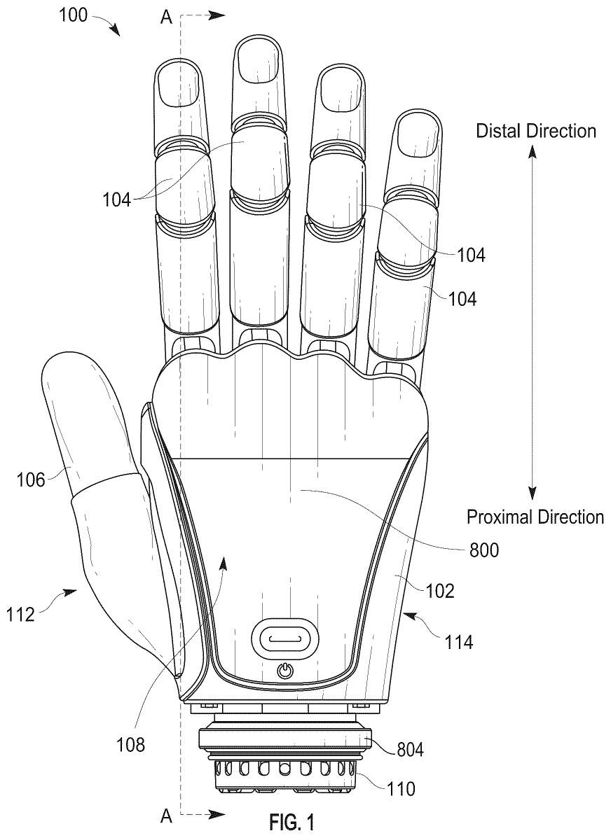

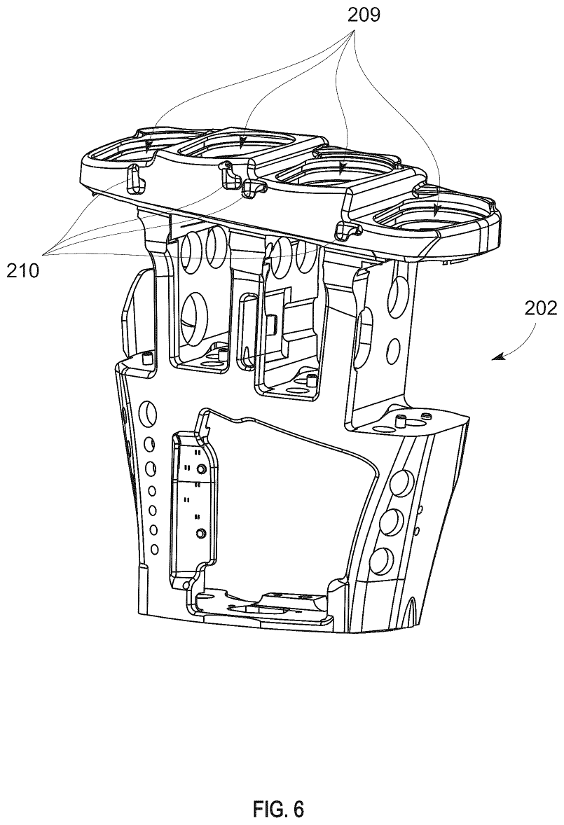

FIG. 1 is a dorsal view of an example of a prosthetic hand 100. The prosthetic hand includes a palm portion 102, four prosthetic digits 104, and a prosthetic thumb 106. The palm portion 102 includes a dorsal side 108, a palm side 128 (see FIG. 8B) opposite the dorsal side 108, a lateral inner side 112 to which the prosthetic thumb 106 is connected, and a lateral outer side 114 opposite the lateral inner side 112. A cover assembly 800 extends over and around the palm portion 102. The cover assembly 800 is formed from various cover portions, as further described herein. The prosthetic hand 100 may be attached to a wrist device 110 as shown. The prosthetic hand 100 may be attached to an upper limb such as a natural or prosthetic arm, e.g., via the wrist device 110. There may be one, two, three or four of the prosthetic digits 104. The prosthetic digits 104 are laterally spaced apart and extend distally from a distal end of the palm portion 102. The prosthetic thumb 106 extends distally from the lateral inner side 112 of the palm portion 102. Each of the prosthetic digits 104 and the thumb 106 may include one or more rotatable segments that are actuated by one or more motors and/or links to move. Each prosthetic digit 104 and the thumb 106 may be actuated by its own respective dedicated motor. Various prosthetic digits or thumbs or features thereof may be used with the prosthetic hand 100, for example those described in U.S. Pat. No. 8,986,395, “HAND PROSTHESIS”, issued on Mar. 21, 2015, in U.S. Pat. No. 9,387,095, “PROSTHETICS AND ORTHOTICS”, issued on Jul. 12, 2016, in U.S. Pat. No. 8,197,554, “ROTARY ACTUATOR ARRANGEMENT”, issued on Jun. 12, 2012, in U.S. Pat. No. 9,99,522, “PROSTHETIC DIGIT FOR USE WITH TOUCHSCREEN DEVICES”, issued on Jun. 19, 2018, in U.S. Pat. No. 11,083,600, “PROSTHETIC DIGIT FOR USE WITH TOUCHSCREEN DEVICES”, issued on Aug. 10, 2021, in U.S. Pat. No. 10,973,660, “POWERED PROSTHETIC THUMB”, issued on Apr. 13, 2021, in U.S. application Ser. No. 17/199,176, “POWERED PROSTHETIC THUMB”, filed on Mar. 11, 2021, in U.S. App. No. 62/599,559, “POWERED PROSTHETIC THUMB”, filed on Dec. 15, 2017, in U.S. application Ser. No. 17/602,247, “PROSTHETIC DIGIT WITH ARTICULATING LINKS”, filed on Oct. 7, 2021, in U.S. App. No. 62/832,166, “PROSTHETIC DIGIT WITH ARTICULATING LINKS”, filed on Apr. 10, 2019, in U.S. application Ser. No. 17/612,539, “ACTUATION SYSTEMS FOR PROSTHETIC DIGITS”, filed on Nov. 18, 2021, in U.S. App. No. 62/850,675, “ACTUATION SYSTEMS FOR PROSTHETIC DIGITS”, filed on May 21, 2019, in U.S. application Ser. No. 17/760,742, “PROSTHETIC DIGITS AND ACTUATORS”, filed on Mar. 15, 2022, in U.S. App. No. 62/902,227, “PROSTHETIC DIGIT ACTUATORS WITH GEAR SHIFTING”, filed on Sep. 18, 2019, in U.S. application Ser. No. 17/098,045, “PROSTHETIC DIGIT ACTUATOR”, filed on Nov. 13, 2020, in U.S. App. No. 62/935,852, “PROSTHETIC DIGIT ACTUATOR”, in filed on Nov. 15, 2019, in U.S. App. No. 63/064,614, “PROSTHETIC DIGIT ACTUATOR”, filed on Aug. 12, 2020, and/or in U.S. Provisional App. No. 63/488,946, titled “PROSTHETIC THUMB WITH ROTATION AND COMPOUND FLEXION,” and filed on Mar. 7, 2023 (Atty Docket #TOUCH.035PR), each of which is incorporated by reference herein in its entirety and forms a part of this specification for all purposes. Thus, the various features described herein, for attachment of digits via filaments and/or for covers for a prosthetic hand, may be used with a variety of different prosthetic digits or thumbs.

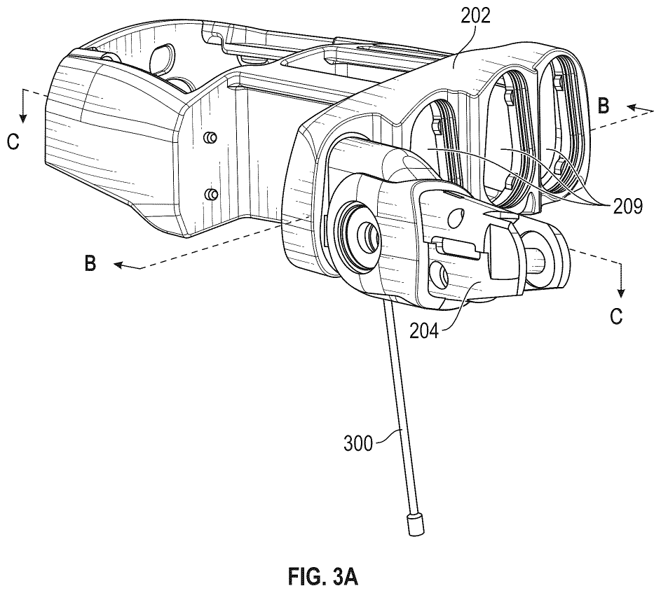

For reference, various reference directions and sides of the prosthetic hand are indicated, as described herein. For example, FIG. 1 is a dorsal side view, FIG. 3B shows a palm side perspective view, etc. Further, the prosthetic hand 100 shown and described herein is a right-sided prosthetic hand 100. Thus, the same features may be incorporated into a mirrored, left-sided prosthetic hand 100, with the appropriate mirroring of the reference directions and sides described herein for a corresponding right-sided prosthetic hand 100.

### B. Filament Attachment of Prosthetic Digits

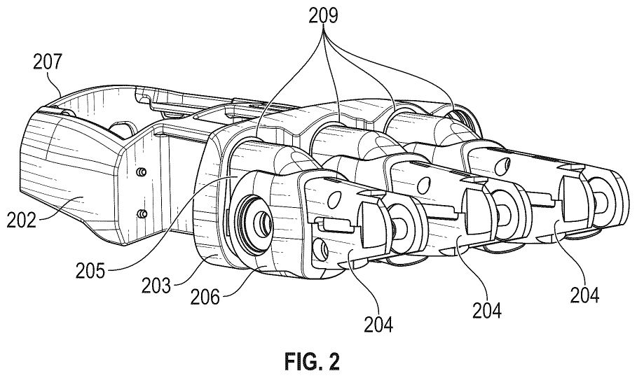

FIG. 2 is a perspective view showing an example of a chassis 202 with components of another embodiment of prosthetic digits 204 attached to the chassis 202. The chassis 202 is included inside, e.g., carried or supported by the palm portion 102 of the prosthetic hand 100 of FIG. 1. The prosthetic digits 204 may have the same or similar features as the prosthetic digits 104, and vice versa. A proximal end of a proximal base 205 of the prosthetic digit 204 is attached to a distal end 203 of the chassis 202. A rotational proximal segment 206 of the prosthetic digit 204 is rotationally attached to a distal end of the proximal base 205. The prosthetic digit 204 may include additional rotational or non-rotational segments. A variety of different types of prosthetic digits may be attached, such as those mentioned above, such that the filament-base securement mechanism described further herein may be agnostic to the type of digit.

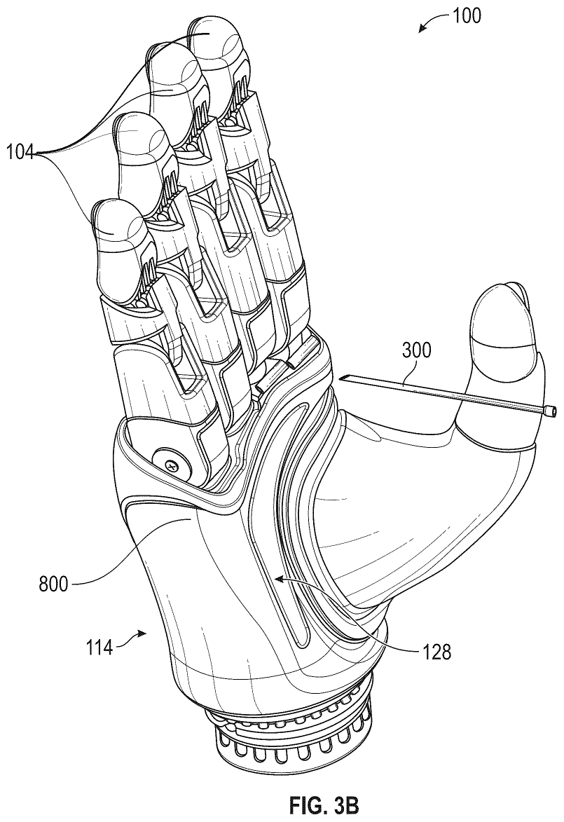

The chassis 202 is an internal structural component of the palm portion 102 of the prosthetic hand 100. The chassis 202 may be located interior of the cover assembly 800, for example within a palm portion thereof, in the assembled prosthetic hand 100. The chassis 202 comprises a rigid structure that extends throughout the inside of the palm portion 102 of the prosthetic hand 100. The chassis 202 may be formed from any suitable material, such as metal (i.e., aluminum, titanium, magnesium, copper, steel, etc.), hard plastics, polymers, other suitable materials, or combinations thereof, to provide structural support to the palm portion 102. The proximal end 207 of the chassis 202 may be secured, for example, to a prosthetic wrist or wrist attachment portion. The distal end 203 of the chassis may comprise one or more (i.e., one, two, three, or four etc.) openings 209 that may receive the corresponding proximal base 205 of the respective prosthetic digit 204. The openings 209 of the chassis 202 may each provide a structural stop (see FIG. 4B) to limit proximal advancement of the proximal base 205 into the chassis 202 beyond a certain location. Proximal advancement may be limited such that recesses in the prosthetic digit 204 and the chassis 202 align, as further described.

FIGS. 3A-4C depict various views of an example of a filament-based securement mechanism for securing the prosthetic digit 204 to the chassis 202. FIGS. 3A and 3B show an example of a filament 300 being inserted into a first groove 208 to secure the prosthetic digit 204 to the chassis 202. The first groove 208 may be one of multiple grooves. The first groove 208 may be a relatively lower or proximal groove among the multiple grooves. In some embodiments, the first groove 208 may be the only groove. FIG. 3A depicts the chassis 202 with part of one prosthetic digit 204 being attached to a first one of the openings 209, while the remaining openings 209 do not have digits attached thereto. FIG. 3B shows a perspective view of the prosthetic hand 100 having the cover assembly 800 and four of the prosthetic digits 104. FIGS. 4A and 4B are rotated cross-section views, with the cross-sections taken along the lines B-B and C-C respectively as shown in FIG. 3A, showing the filament 300 inserted within the first groove 208. FIG. 4C is a closeup detail view of the first groove 208 as taken from FIG. 4B as indicated.

As shown in FIGS. 3A-4C, the first groove 208 may be formed between the proximal end of the prosthetic digit 204 and a corresponding opening of the chassis 202. The first groove 208 is formed with the prosthetic digit 204 and the chassis 202 assembled together. The first groove 208 may encircle an outer perimeter of the proximal end of the prosthetic digit 204 and a corresponding inner perimeter of the opening 209. The filament 300 may be inserted into the first groove 208 in order to secure the prosthetic digit 204 with the chassis 202. A user may locate and insert the filament 300 into the first groove 208 via an access opening 210 of the chassis 202. The access opening 210 may be cylindrical. The access opening 210 extends to the groove 208. The access opening 210 may be located on a palm-side of the chassis 202. The access opening 210 may be on a dorsal-side of the chassis 202, or in other locations. There are four access openings 210 corresponding to the four openings 209 to attach each of the four prosthetic digits 204.

The first groove 208 extends annularly through, and is formed by complementary recesses 216, 214 in the chassis 202 and the digit 204, respectively (see FIG. 4B). The first recess 214 extends annularly around, and is formed within an outer surface 224 of, the proximal end of the prosthetic digit 204, for example at a proximal end of the proximal base 250. The second recess 216 extends annularly inside, and is formed within an inner surface 222 of, the distal end of the chassis 202. The first and second recesses 214, 216 align to form the complete first groove 208.

The first groove 208 extends along an annular path. “Annular” as used herein has its usual and customary meaning, and includes without limitation rounded, circular, circuitous, circumferential, around a perimeter, etc. In some embodiments, the first groove 208 may have an oblong shape as shown in FIG. 4A. The first groove 208 comprises two opposing straight sides 208A and two opposing curved sides 208B as shown in FIG. 4A. A palm side of the first groove 208 may have a relatively wider curved side 208B than the opposite curved side 208B at a dorsal side of the first groove 208 as shown, or vice versa. In some embodiments, the first groove 208 may be circular, oval, elliptical, racetrack-shaped, trapezoidal, or other annular shapes.

The first groove 208 extends back onto itself to form a continuous through-hole or pathway such as a continuous “tunnel.” A distal end 228 of the first groove 208 thus extends to and is open to a proximal region 226 of the first groove 208. The distal end of the first groove 208 may open to other portions of the first groove 208, such as more central locations or other regions along the length of the first groove 208 located farther distally of the proximal region 226. The distal end 228 of the first groove 208 is curved and the proximal region 226 is straight. The distal end 228 and/or proximal region 226 of the first groove 208 may be straight, curved, or other shapes. The filament 300 extends through the first groove 208 to fill the entire length of the first groove 208. The filament 300 may have a length that is less than or greater than the length of the first groove 208. The filament 300 may have an end portion 306 that fills or partially fills the access opening 210 when the filament 300 is fully inserted into the first groove 208, such that a user can remove the filament 300 from the first groove 208 via the end portion 306 when the digit needs to be removed and/or replaced. The end portion 306 may be a ferrule attached to the filament 300 and inserted into the first groove 208, such that the end portion 306 is flush with an access opening of the chassis 202 leaving no protrusion. The filament 300 may alternatively have no end portion 306. The filament 300 may have a uniform cross-section along its entire length. The first groove 208 may be shorter, such that the distal end 228 is not open to the proximal region 226 or to another portion of the groove 208, and thus form a “blind” hole. The first groove 208 extends along a path that is within a plane, as shown. The tunnel of the first groove 208 defines a central axis that lies within the plane. The plane is perpendicular to the distal and proximal directions of the chassis 202. The plane may be at an angle to the distal and proximal directions. The path of the first groove 208 may not be planar. The first groove 208 may extend in the proximal and/or distal directions relative to the location of the access opening 210, e.g., in a spiral shape, or with a step down and/or step up along its path, etc.

FIG. 4C shows a closeup of the recesses 214, 216. The recess 214 has a first side 214A that is perpendicular to a second side 214B, which is perpendicular to a third side 214C. The second side 214B, which may be a long side, is relatively longer than each of the first side 214A and second side 214B, the latter of which may be short sides. The recess 214 thus has a three-sided cross-section defining an opening that faces in an exterior direction away from the recess 214. The recess 216 has a first side 216A, a second side 216B, and a third side 216C, which have a similar arrangement as described for the three sides of the recess 214. The resulting first groove 208 therefore has a cross-section with four sides, e.g., a square as shown. The first groove 208 has a closed cross-section due to the chassis 202 abutting the prosthetic digit 204 along a first interface 218 and along an opposite second interface 220. The interfaces 218, 220 are areas of contact of opposing outer surfaces of the chassis 202 and prosthetic digit 204 on opposing sides of the first groove 208. The filament 300 is therefore completely enclosed within the first groove 208. The first groove 208 may have a uniform cross-section, for example the cross-section as shown, along most or all of the length of the first groove 208. The first groove 208 leads to the access opening 210, which has a circular cross-section.

In some embodiments, the recesses 214, 216 and thus the resulting first groove 208 may have different shapes and sizes. The first groove 208 may be rectangular, or circular, or have three, five, six, seven, eight or more sides, and/or have a cross-section with a non-uniform size and/or shape along the length of the first groove 208. The first groove 208 may have fillets at the intersections of the sides of the recesses. The access opening 210 may have the same or different cross-sectional shape and/or size as the first groove 208.

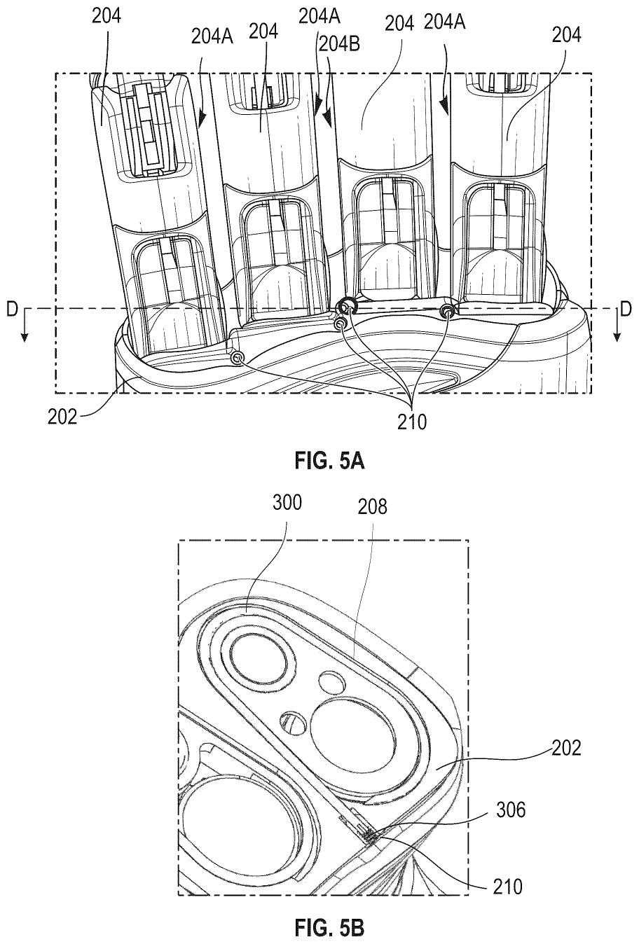

As described further with respect to FIGS. 7 and 8, in some embodiments, the first groove 208 may be used to house a digit sealing ring 750 and the filament 300 may be inserted into a second groove 238. The second groove 238 may be an upper or more distal groove relative to the first groove 208. In some embodiments, there may only be the second groove 238 and no first groove 208. The second groove 238 may have the same or similar features as the first groove 208 discussed above to allow the filament 300 to be inserted into the second groove 238 in the same manner as described with respect to the embodiments where the filament 300 is inserted into first groove 208. The digit sealing ring 750 may provide a waterproof seal between the chassis 202 and the digits 204 to prevent liquid from entering the prosthetic hand 100. The digit scaling ring 750 may be an O-ring. The digit scaling ring 750 may be inserted into the second groove 208 inside the digit opening of the chassis prior to attachment of the digit (e.g., see FIG. 7). The digit sealing ring 750 may extend completely along the entire length of the first groove 208. The first groove 208 may have a relatively longer height in the proximal/distal direction as compared to the second groove 238.

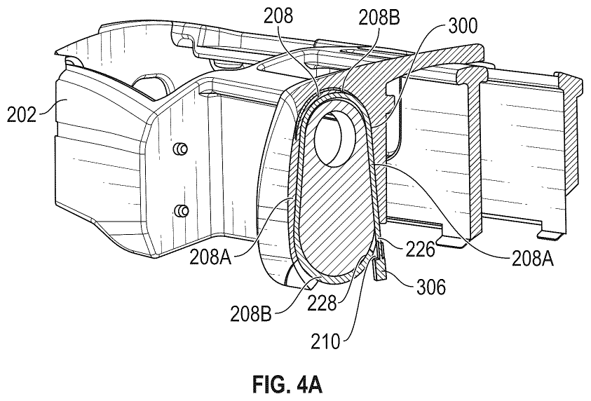

FIG. 5A is a palm-side view of part of the prosthetic hand 100 showing an example embodiment of the access openings 210. The access openings 210 lead to the grooves 208. The access openings 210 may have any shape that is configured to receive the filament 300, for example, a circular shape as shown, or an ovular shape, and square shape, a rectangular shape, etc. The access openings 210 are formed in a distal end of the chassis 202. The access openings 210 are located adjacent to the proximal end of the respective proximal digit 204 to which the filament 300 within the corresponding first groove 208 or second groove 238 is secured. The access openings 210 for the first, second and fourth prosthetic digit 204 (i.e., pinky, ring, and pointer digit, respectively) may be located on the lateral inner side 204A of the respective digit. The access opening 210 for the third prosthetic digit 204 (i.e., middle digit) is on the lateral outer side 204B of the third prosthetic digit. The access openings 210 may have the reverse positions, or variations thereof, and/or have central locations relative to the respective digit. The access openings 210 may face the palm side, such that the access openings 210 extend axially perpendicularly to axes extending along the respective prosthetic digit 204. The access openings 210 may extend along and face in other directions, such as distally, or dorsally, or in the lateral inner or outer directions, or at an angle to any of these directions.

FIG. 5B is a rotated partial cross-section view of the first groove 208 for the index digit 204 (next to the thumb), as taken along line C-C in FIG. 5A. The filament 300 fully surrounds the circumference of the proximal base 205.

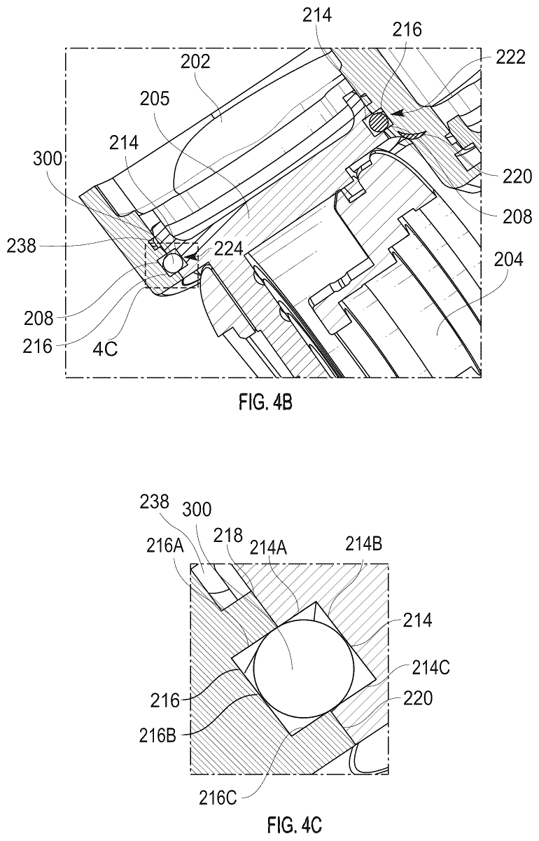

FIG. 6 shows another embodiment of a chassis 202. While FIGS. 3A-5B are shown and described with respect to an embodiment of the chassis 202 where the access openings 210 are located on the palm side of the chassis 202, in other embodiments, as mentioned, the access openings 210 may be located on the dorsal side of the chassis 202. An example of such embodiment is shown in FIG. 6. The same features and functions as described herein with respect to the embodiments of the chassis having the access openings 210 on the palm side may apply to the embodiments of the chassis having the access openings 210 on the dorsal side. Thus, for example, the structure and description for the various features of the chassis 202, the filament 300, the first groove 208, the second groove 238, and the recesses 214, 216 and how they are operated for securing the prosthetic digits 204 to the prosthetic hand 100, may apply to embodiments where the access openings 210 are located on the dorsal side of the chassis 202 in the same way that they apply to embodiments where the access openings 210 are located on the palm side of the chassis 202, and vice versa.

As shown in FIG. 6, there may be four access openings 210 distributed laterally along the dorsal side of the chassis 202. The access openings 210 may be distributed non-uniformly, as shown, for example with the two middle access openings 210 closer to each other than to the two respective adjacent outer openings. In some embodiments, the access openings 210 may be distributed uniformly. The access openings 210 may be located on laterally inward sides of each opening 209 of the chassis 202, as shown. In some embodiments, the access openings 210 may be located on laterally outward sides of each opening 209 of the chassis 202. The access openings 210 may be at varying distal and proximal locations relative to each other, or all or some of them may be at the same relative distal/proximal locations. The access openings 210 may have L-shaped openings as shown, or other shapes such as circular, triangular, rectangular, etc.

FIG. 7 is a close up view of one of the openings 209 of the chassis 202, showing embodiments of the second groove 238 and the first groove 208. For clarity, the chassis 202 is shown without the prosthetic digit 204 and the second groove 238 is shown without the filament. As shown, the first groove 208 may be configured to receive the digit scaling ring 750 therein, and the second groove 238 may be configured to receive the filament 300 therein, as discussed above with reference to FIG. 4C.

FIG. 8A shows a partial cross-section view of the index digit 204 (next to the thumb), as taken along line A-A in FIG. 1. FIG. 8B is a close up detail view of the first groove 208 and the second groove 238 as taken from FIG. 8A as indicated. The filament 300 is shown in the second groove 238 and the digit sealing ring 750 is shown inserted into the first groove 208.

As discussed above, in some embodiments, the second groove 238 may be formed by complementary recesses 216, 214 in the chassis 202 and the prosthetic digit 204, respectively. When the filament 300 is inserted into the second groove 238, a portion of the filament 300 is located in the recess 216 of the chassis 202 and the remainder of the filament is located within the recess 214 of the digit 204 such that the filament 300 secures the chassis 202 and the digit 204 together, preventing axial movement of the digit 204, as described herein.

The digit sealing ring 750 may provide a seal, such as a waterproof and/or airtight seal, between the chassis 202 and the digit 204. The sealed interface may prevent fluid from traversing the interface in the proximal direction from the digit into the lower part of the chassis. The digit sealing ring 750 may be compressed, e.g., radially compressed, to provide such seal, and to allow for removal or insertion of the digit into the opening 209 of the chassis 202. In some embodiments, a portion of the digit sealing ring 750 in an unconstrained state (e.g., prior to insertion of the digit) may be located within the first groove 208, and the remainder of the digit sealing ring 750 may protrude radially inward and outside of the first groove 208. Upon insertion of the prosthetic digit 204, the inwardly protruding portion of the sealing ring 750 may be compressed radially outwardly into the first groove 208 by the proximal base 205 of the prosthetic digit 204. In some embodiments, the first groove 208 may be formed by corresponding and aligning recesses of the prosthetic digit and the proximal base, for instance as described with respect to the recesses forming the second groove 238.

As described above, in some embodiments, the filament 300 may be located in the first groove 208, which may be a lower or more proximally located groove. In such embodiments, the first groove 208 is formed by the complementary recesses 216, 214 of the chassis 202 and the prosthetic digit 204, respectively. After the filament 300 is inserted into the access opening 210, a portion the filament 300 is located in the recess 216 of the first groove 208 of the chassis 202 and the remainder of the filament is located within the recess 214 of the digit 204 such that the filament 300 secures the chassis 202 and the digit 204 together, preventing axial movement of the digit 204. In such embodiments, the digit scaling ring 750 may be located in the second groove 238, which may be an upper or more distally located groove, of the chassis 202 and compressed into the prosthetic digit 204 in order to provide a waterproof seal between the chassis 202 and the digit 204. The digit sealing ring 750 may be compressed further to allow for removal or insertion of the digit into the opening 209 of the chassis 202.

The features and descriptions of embodiments where the filament 300 is located in the second groove 238 and the digit sealing ring 750 is located in the first groove 208 may apply to embodiments where the access openings 210 are located on the palm side of the prosthetic hand 100 as well as embodiments where the access openings 210 are located on the dorsal side of the prosthetic hand 100. Similarly, features and descriptions of embodiments where the filament 300 is located in the first groove 208 and the digit sealing ring 750 is located in the second groove 238 may apply to embodiments where the access openings 210 are located on the palm side of the prosthetic hand 100 as well as embodiments where the access openings 210 are located on the dorsal side of the prosthetic hand 100.

The filament 300 forms an elongated cord extending along a longitudinal length. The filament 300 may be uniform in cross-section along the entirety of its longitudinal length. The filament 300 may have sufficient stiffness to be inserted into and pushed through the groove 208. For example, there may just be the elongated portion without any end features.

The filament 300 may include an end portion 306. The end portion 306 may aid in inserting the filament 300 into the access opening 210 and through the first groove 208. The end portion 306 may have a larger width than the filament 300. The end portion 306 may be a cylindrical end cap or ferrule configured to be received into the access opening 210. The access opening 210 may have a depth corresponding to the longitudinal length of the end portion 306. In some embodiments, the first groove 208 may have a smaller width than the access opening 210, such that a distal edge, e.g. palm side-facing edge, within the access opening 210 at the interface with the groove 208 contacts a distal end of the end portion 306, and thereby limits distal travel of the filament 300 farther into the groove 208. In some embodiments, the second groove 238 may have a smaller width than the access opening 210, such that a distal edge, e.g. dorsal side-facing edge, within the access opening 210 at the interface with the groove 208 contacts a proximal end of the end portion 306, and thereby limits distal travel of the filament 300 farther into the first groove 208. The end portion 306 may be secured within the access opening 210, e.g., via a friction fit with the access opening 210, threaded engagement, or other means.

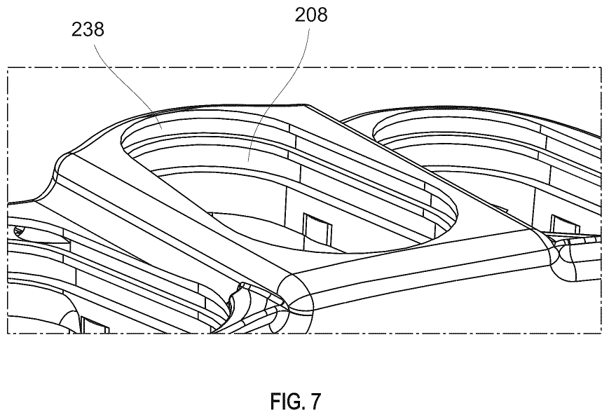

FIG. 9 is a perspective, partial cross-section view of an example embodiment of the filament 300 having a wound construction. A portion of the filament 300 (at the right end, as oriented) is shown in cross-section. The wound construction includes a winding 302 wrapped in a spiral around a longitudinally-extending core 304. The winding 302 and the core 304 may be metallic (e.g., bronze, steel, copper, titanium, etc.) and made of the same or different materials. The filament 300 may be metallic, polymeric, plastic, other materials, or combinations thereof. The winding 302 may be formed from a strand having a smaller diameter than the core 304. In some embodiments, the winding 302 may be formed from a strand with a diameter from 0.25 mm to 0.40 mm, from 0.30 mm to 0.35 mm, e.g., 0.31 mm, 0.32 mm, 0.33 mm, 0.34 mm, or ranges between such values, and the like. In some embodiments, the core 304 may have a diameter from 0.3 mm to 0.55 mm, from 0.40 mm to 0.45 mm, e.g., 0.41 mm, 0.42 mm, 0.43 mm, 0.44 mm, etc., ranges between such values, and the like.

The overall diameter of the filament 300 may be, for example, from 0.75 mm to 1.25 mm, from 1 mm to 1.05 mm, e.g., 1.01 mm, 1.02 mm, 1.03 mm, 1.04 mm, or ranges between any of these values, and the like. In embodiments where the filament 300 has a wound construction, a portion of the core 304 may protrude longitudinally farther out from an end of the winding 302. This may allow a user to easily insert or remove the filament 300 into or out of the groove 208. The filament 300, whether wound or other construction, may have a length from 30 millimeters (mm) to 90 mm, from 40 mm to 80 mm, from 50 mm to 70 mm, from 55 mm to 65 mm, about 65 mm, or 65 mm. This length can be increased/decreased to hold a larger/smaller object in a different sized hole. The length of the filament may be custom sized in thickness and/or length for a particular width and length of the groove 208.

In some embodiments, the filament 300 may have an end portion 306 as shown in FIG. 5A. The end portion 306 may enable the user to easily insert or remove the filament 300 into or out of the groove 208. The end portion 306 may be a ferrule, a cap, a rigid piece, a fitting, a bracket, etc. The end portion 306 may be attached to the filament 300 via crimping, bonding, soldering, screwing, other methods, or combinations thereof. In other embodiments, the filament 300 may have no end portion 306. The filament 300 may have a wound construction of uniform cross-sectional area along its entire length, as mentioned. The wound construction may allow for even distribution of load over the material, preventing the filament 300 from being damaged and/or stuck as the filament 300 is fit into the first groove 208 or second groove 238, e.g. for a friction force fit. The wound construction may also be more flexible, as compared to a single core metal material or a more rigid material, so the wound construction is less likely to damage the chassis 202 during fitting. The filament 300 may conform to the first groove 208 or second groove 238 to allow easy insertion and to track around the first groove 208 or second groove 238 with minimal force. The wound construction may provide a more durable filament 300 such that it can pass high force requirements and resist deformation, which allows for removal of the filament 300 without damaging the chassis. For applications with lower loads, other non-wound constructions of the filament 300 may be used.

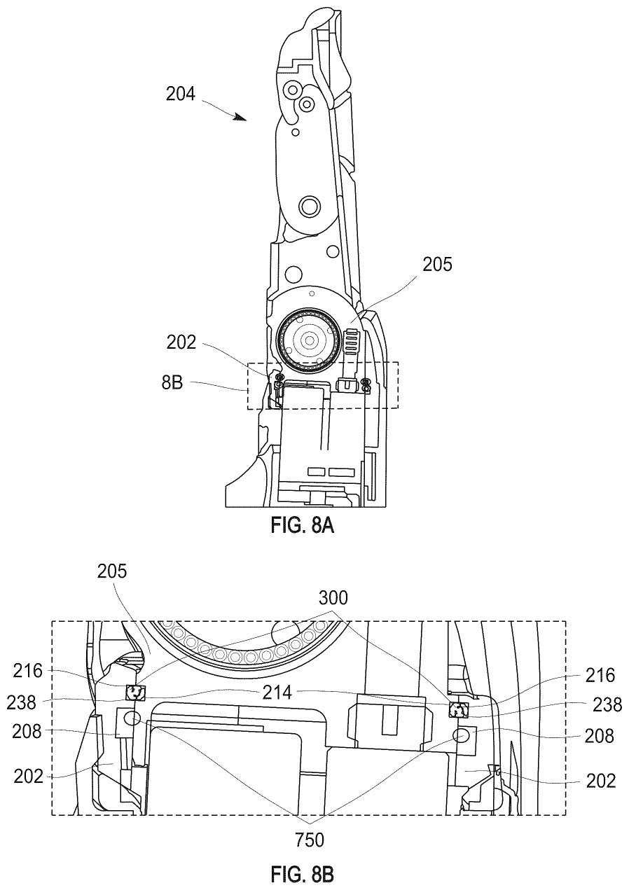

FIG. 10 is a flow chart illustrating an example method 500 of attaching a prosthetic digit to a prosthetic hand. The method 500 begins with step 501, where the prosthetic digit 204 is assembled with (e.g., by friction fit) the chassis 202. A proximal end of the prosthetic digit 204 may be received into the chassis opening 209. The proximal end of the prosthetic digit 204 may be laterally constrained by the opening 209 of the chassis 202, e.g., in the palm and dorsal directions, and in the lateral inner and lateral outer directions.

The method 500 then moves to step 502 where the first and second recesses 214, 216 of the prosthetic digit 204 and the chassis 202 are aligned to form the annular second groove 238. In some embodiments of the method 500, the first groove 208 may be used instead of the second groove 238, and any description herein of the method 500 with respect to the second groove 238 may apply to the first groove 208. Step 502 may include a user locating the second groove 238 via the access opening 210 of the chassis 202. The recesses 214, 216 may be aligned as shown and described herein, e.g., with respect to FIGS. 3A-5B.

The method 500 then moves to step 503, where the user inserts the filament 300 into the second groove 238. The filament 300 is pushed through the access opening 210 and follows the enclosed groove 208 formed when the base of the prosthetic digit 204 and the distal end of the chassis 202 are assembled. As the filament 300 is pushed farther into the groove 208, the filament 300 extends around the base 205 of the prosthetic digit 204, forming complete fixation around the circumference of the base 205. The prosthetic digit 204 is axially constrained by the filament 300 such that movement of the prosthetic digit 204 in the distal direction is countered via the filament 300 interacting with the groove 208. The filament 300 may prevent relative movement at the first and second interfaces 218, 220 (see FIG. 4C). In some embodiments, the groove 208 may be filled with an adhesive to more strongly hold and/or seal the prosthetic digit 204 with the chassis 202. The filament 300 may fill the entire length of the groove 208, or a portion thereof. The cross-section of the filament 300 may occupy the entire cross-section of the groove 208, or a substantial majority thereof. The filament 300 may be inserted into the groove 208 as shown and described herein, e.g., with respect to FIGS. 3A-9.

The filament securement mechanism disclosed herein provides for fully supported fixation along the entire groove 208 between the first interface 218 and opposite second interface 220. The filament securement mechanism further allows for a tunable breakaway, as different filament 300 materials and groove 208 dimensions can be used, and thus the strength of the joint can be tightly controlled. This advantageously allows for a prosthetic joint to be engineered to deliberately fail in an extreme load event to protect the device and/or user. Due to its simplicity, the filament 300 securement allows for easy infield replacement. The filament securement mechanism allows for a digit 104 to be removed and reattached with a single component (i.e., the filament 300). For example, the use of the filament 300 makes it possible to replace the prosthetic digit 204 without the need for a hand tool. This may allow the user of the prosthetic hand 100 to remove and/or attach the prosthetic digits 204 themselves as compared to other devices with more complicated digit attachment mechanisms, which often require the user to seek assistance from a care provider to attach and/or remove prosthetic digits from their prosthetic hand. Filament fixation according to the present disclosure is also vibration-proof, meaning that even if the filament 300 moves, the prosthetic digit 204 remains secured with the chassis 202. The filament fixation further provides for minor flexibility of conforming under load, e.g., when the filament is made from a flexible, durable material, such as rubber.

### C. Waterproof Cover for Prosthetic Hand

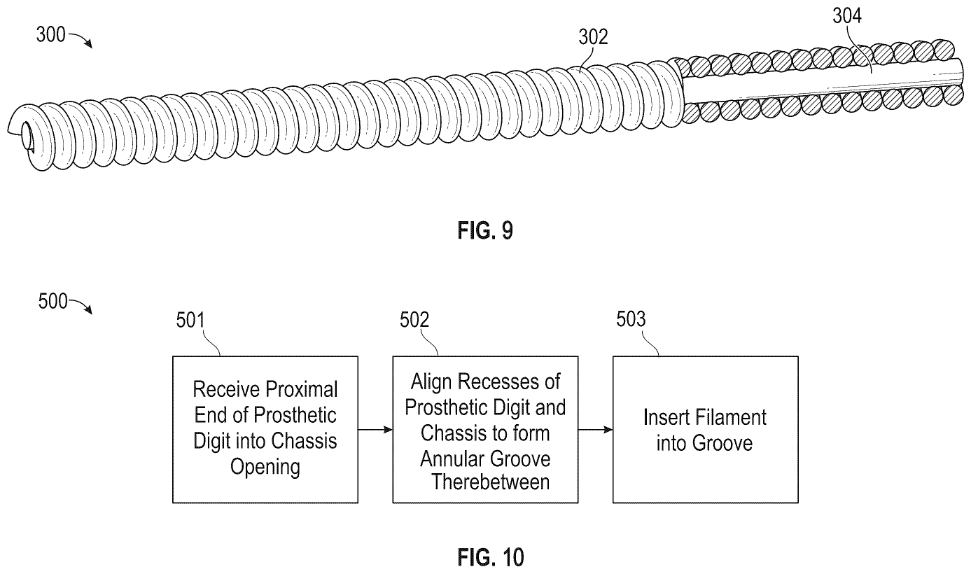

FIGS. 11A and 11B are perspective dorsal and perspective palm side views respectively of the prosthetic hand 100 with the hand cover assembly 800. The cover assembly 800 includes a palm cover 700, a thumb cover 702, and a dorsal cover 710. FIG. 9 shows an exploded view of an example of the thumb cover 702, a thumb clamp 1004, and the palm cover 700. The hand cover assembly 800 may be waterproof, as further described. The various waterproof features may be impervious or substantially impervious to water and other liquids. The prosthetic hand 100 may include the filament and other features as described herein with respect to FIGS. 1-10 as well as any of the cover features described herein with respect to FIGS. 11A-19. As shown in FIG. 11A, the prosthetic hand 100 may include the wrist device 110, as mentioned, for attaching to a socket or arm. The prosthetic hand 100 in FIG. 11B is shown without the wrist device 110.

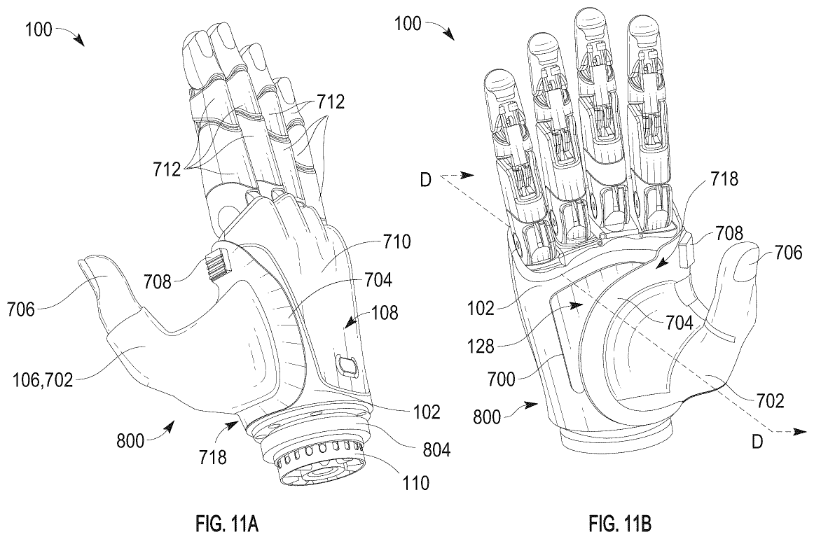

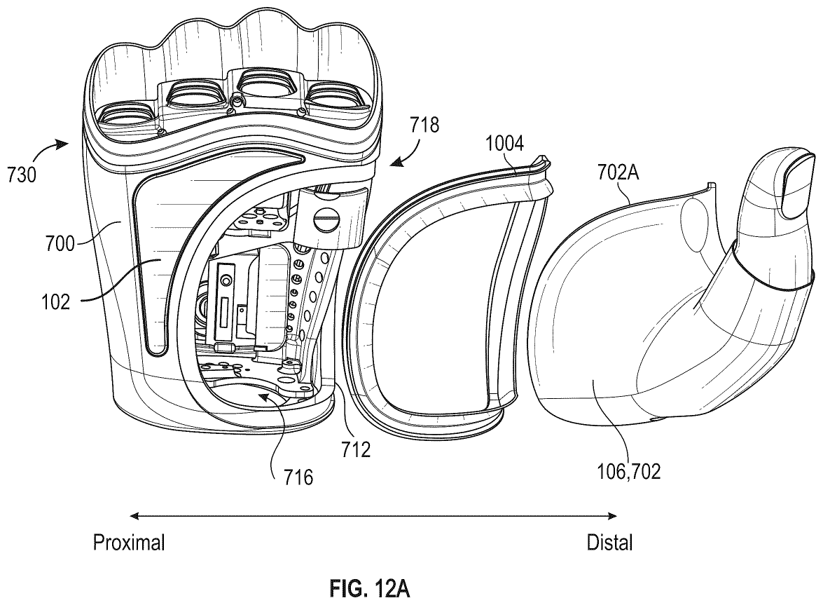

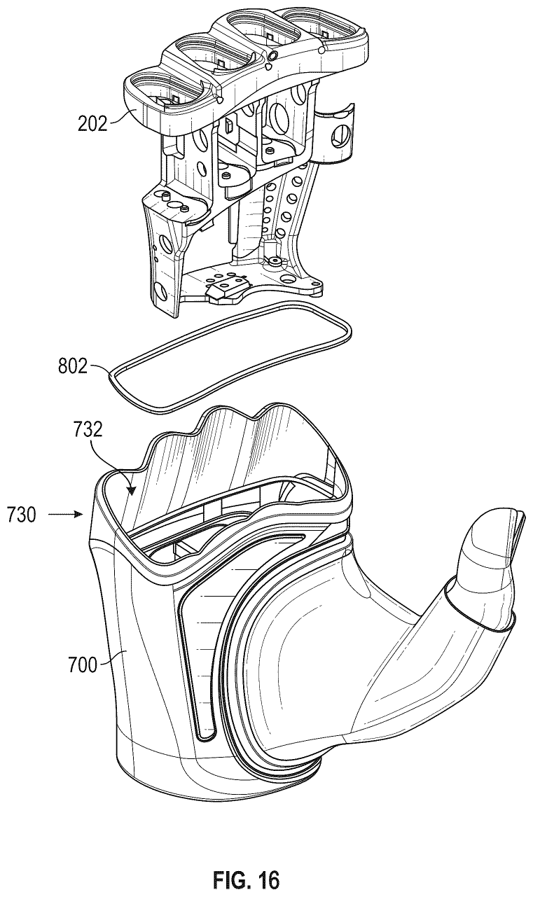

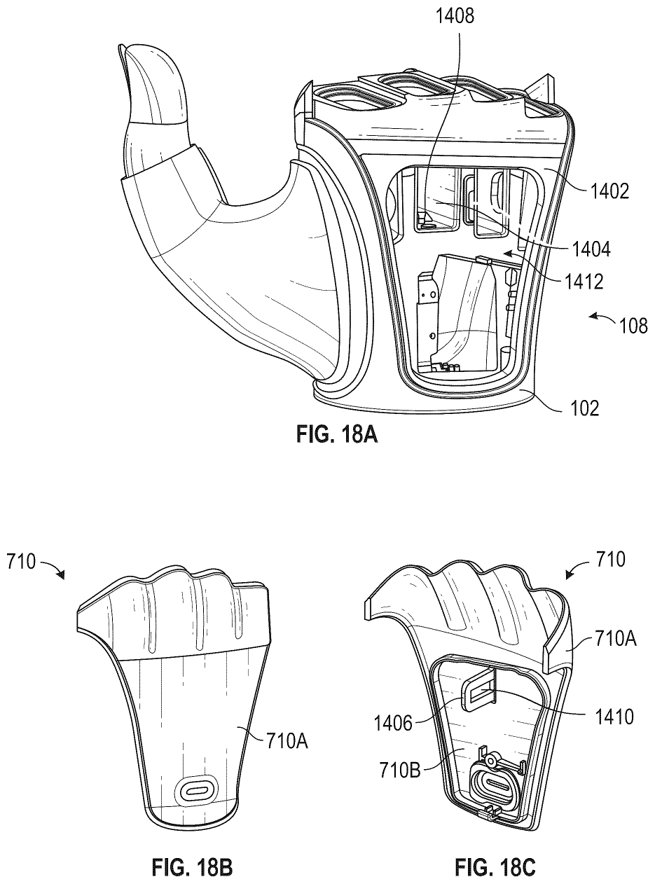

Referring to FIGS. 11A-12B, 16 and 18A-C, the palm cover 700 extends around and covers a portion of the palm portion 102. The palm cover 700 defines a lateral opening 716 (see FIG. 12A) on the lateral inner side 112 of the palm portion 102. The palm cover 700 defines a distal opening 732 (see FIG. 16) on the distal end 730 of the palm portion 102. The palm cover 700 defines a dorsal opening 1412 (see FIG. 18A) on the dorsal side 108 of the palm portion 102. The dorsal cover 710 attaches over the dorsal opening 1412, the thumb cover 702 attaches over the lateral opening 716, and the chassis is inserted through the distal opening 732, as further described. The palm cover 700, such as the dorsal cover 710, may cover the access openings 210 in embodiments where the access openings 210 are located on the dorsal side of the chassis 202.

FIG. 12B is a perspective view of the thumb cover 702 in isolation. The thumb cover 702 includes a sidewall 720. The sidewall 720 defines a closed cross-section. The sidewall extends from a proximal thumb opening 720A to an opposite distal thumb opening 720B. The proximal thumb opening 720A attaches to the lateral opening 716 of the palm cover 700. The prosthetic thumb or portions thereof extend through the distal thumb opening 720B. The proximal thumb opening 720A has a larger area than the distal thumb opening 720B. The sidewall 720 may decrease in width from the proximal thumb opening 720A in the distal direction, and vice versa.

The sidewall 720 of the thumb cover 702 may have multiple layers. As shown, the sidewall 720 may comprise a three-layer textile structure. The thumb cover 702 has elasticity and allows for the flex and rotation movement of the thumb while still being waterproof. The layers may be impervious to water and other liquids or fluids. An internal layer 722 of the sidewall 720 (e.g., on an inner side of the sidewall 720) is made of elastane or similar materials (e.g., Lycra) which provides for good movement interactions with the internal mechanics of the prosthetic thumb. A middle layer 724 of the sidewall 720 (e.g., between inner and outer layers of the sidewall 720) is a rubberized impermeable or waterproof material (e.g., urethane). The middle layer 724 may have a heat activated glue that allows the middle layer 724 to bond with the internal and external layers once heated under pressure. An external layer 726 of the sidewall 720 (e.g., an outer layer of the sidewall 720) is made of a high-performance non-woven composite fabric material suitable for use in high-strength, low-weight applications (i.e., Cuben Fiber, DYNEEMA®, etc.). The external layer 726 is constructed from a thin sheet of ultra-high-molecular-weight polyethylene (UHMWPE) laminated between two sheets of polyester. In some embodiments, the external layer 726 may further include Nylon or a knit mix. The middle layer 724 is located between the internal layer 722 and the external layer 726. The internal layer 722 faces radially inwardly, e.g., toward the thumb. The external layer 726 faces radially outwardly, e.g., away from the middle layer 724. In some embodiments, the thumb cover 702 may include only a single layer, or two, four, five or more layers. In multi-layer designs, the layers may be attached together only along outer perimeters thereof, and/or along inner surfaces thereof, or combinations thereof. In some embodiments, there may be additional layers inwardly of the internal layer 722 and/or outwardly of the external layer 726. In some embodiments, there may be additional layers other than the middle layer 724 between the internal layer 722 and the external layer 726.

The prosthetic thumb 106 may include a thumb tip 706, which may be made from rubberized silicone or any suitable alternative materials. The thumb tip 706 may be detachable and/or replaceable. The cover assembly 800 may further comprise digit covers 712 covering some or all of the prosthetic digits 204. The digit covers 712 may be made of the same and/or similar materials as the other components of the cover assembly 800. In some embodiments, the digit cover 712 may have a silicon rubber sleeve on one or more phalanges of one or more of the prosthetic digits 204 in order to improve grip. The digit covers 712 may be on dorsal and/or palm sides of the prosthetic digits 204.

As shown in FIGS. 11A-11B and 12A, the thumb cover 702 is attached to the palm cover 700 via a thumb clamp 1004 along a thumb interface 718. The thumb clamp 1004 is elongated with a ring shape. The thumb clamp 1004 may be made of a hard plastic such as a polycarbonate blend, or any other suitable alternative. An adhesive may be applied to interior and exterior sides of the thumb clamp 1004 to bond with the thumb cover 702 and the palm cover 700. The thumb clamp 1004 may be attached to the thumb cover 702 and the palm cover 700 using adhesive, mechanical attachments, or combinations thereof. The thumb clamp 1004 secures the thumb cover 702 with the palm cover 700 along the thumb interface 718 over a circumference of the proximal thumb opening 720A of the thumb cover 702. A thumb seal 704 may then be placed on an exterior along the thumb interface 718 over the thumb clamp 1004 and/or over a proximal end of the sidewall 720 of the thumb cover 702. The thumb seal 704 is impermeable to fluid. The cover thus prevents water and other fluids from penetrating the prosthetic hand 100 along the interface or through the thumb cover 702. The thumb seal 704 is made of a hard plastic such as a polycarbonate blend, or any other suitable alternative.

As shown in FIGS. 1 and 11A, the prosthetic hand 100 may include a wrist seal 804. The wrist seal 804 may provide a sealing interface between the prosthetic hand 100 and the wrist device 110. The wrist seal 804 may provide a sealing interface between the prosthetic hand 100 and/or wrist device 110, and a socket, arm, or other component to which the wrist device 110 may be attached. The wrist seal 804 may extend along an outer perimeter of the wrist device 110, such as a distal portion thereof. The wrist scal 804 may extend annularly and completely around the wrist device 110 to completely seal off the path. The wrist seal 804 may be made from a compliant material (e.g., rubber, silicone, etc.) and provide a waterproof seal between the wrist device 110 and other components.

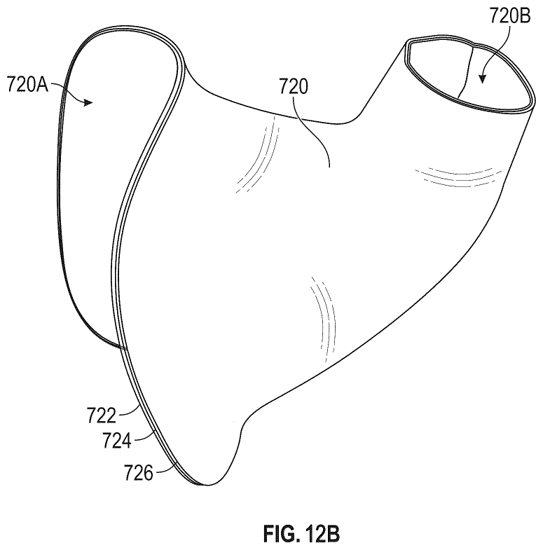

FIG. 13 is a perspective view of an embodiment of a grip pad 708 with the thumb tip 706 pressed against the grip pad 708. The thumb seal 704 at a distal end thereof may include the grip pad 708. The grip pad 708 is compliant and/or collapsible. The grip pad 708 may be deformable. In some embodiments, the grip pad 708 may be filled with air. In some embodiments, the thumb seal 704 may not include the grip pad 708.

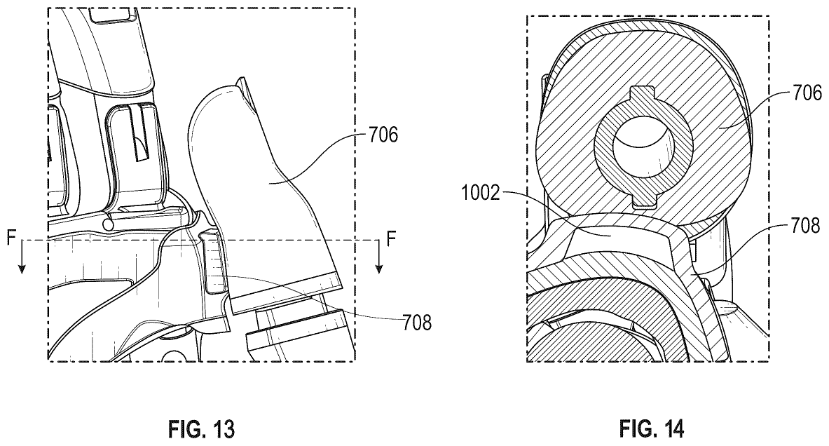

FIG. 14 is a cross-section view of the grip pad 708 and thumb tip 706 as taken along the line F-F in FIG. 13, showing an air-pocket 1002 within the grip pad 708. When the thumb tip 706 presses against the grip pad 708, the central portions of the laterally outer surface of the grip pad air-pocket 1002 collapses, providing enhanced gripping as there are higher pressure points on the corners of the grip pad 708 which reduces the likelihood of slipping and/or rotation. This advantageously enhances the ability of a user to grip thin objects between the prosthetic thumb and grip pad 708. The grip pad 708 may be made from silicone, a mix between silicone and a Thermoplastic polyurethane (TPU), or other similar materials. The surface of the grip pad 708 may be textured or smooth. In some embodiments, having a grip pad 708 with a smooth surface is more desirable for use in wet environments.

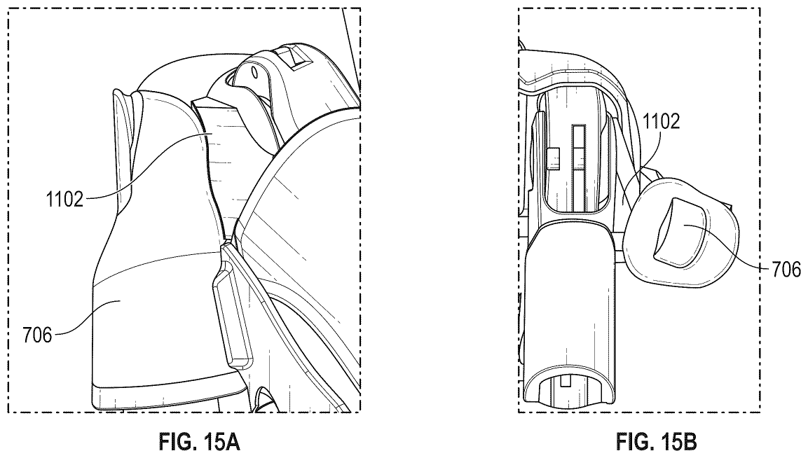

FIGS. 15A and 15B show perspective views of a detachable thumb attachment 1102 included in some embodiments of the present disclosure. The detachable thumb attachment 1102 may be adhered to the thumb seal 704 above the grip pad 708. The detachable thumb attachment 1102 may be an angular protrusion. The thumb tip 706 may include a complimentary recess that is designed to receive the detachable thumb attachment 1102. The detachable thumb attachment 1102 may advantageously allow for a firmer grip.

FIG. 16 is an exploded view of part of the prosthetic hand 100, showing the palm cover 700, the chassis 202, and a palm sealing ring 802. Conventionally, for prosthetic hand assembly, prosthetic digits are first secured with a structural portion of a palm of a prosthetic hand before a cover is attached. In embodiments according to the present disclosure, the palm cover 700 and chassis 202 are assembled together prior to attachment of the prosthetic digits with the chassis 202. This advantageously allows for simpler attachment and removal of the prosthetic digits, and for the prosthetic hand 100 to have a cleaner look with no visible fasteners, and requires fewer parts for assembly.

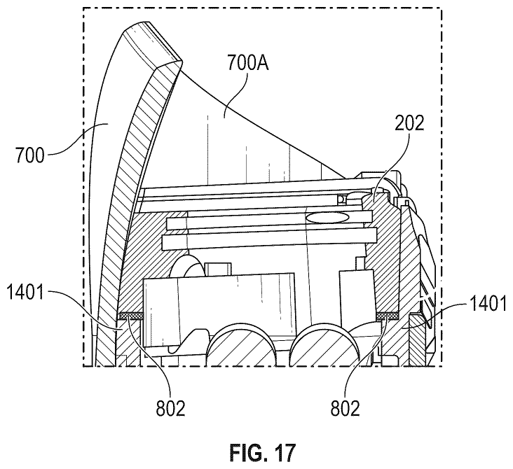

FIG. 17 is a cross-section view of the prosthetic hand 100 as taken along the line E-E in FIG. 11B and showing the arrangement of the palm cover 700, the chassis 202, and the palm sealing ring 802 when assembled. The cover has a ridge 1401 extending along at least a portion of an inner surface of the palm cover. The ridge 1401 may extend completely around the inner surface 700A of the palm cover 700. The palm sealing ring 802 is compressed between the palm cover 700 and the ridge 1401 in order to prevent fluid from penetrating the palm cover 700. The palm sealing ring 802 may thus extend along an inner palm side of the chassis 202 as well as an inner dorsal side of the chassis 202 along the ridge 1401. The palm sealing ring 802 may therefore extend along more than just a palm side of the interior of the chassis 202. The palm sealing ring 802 may be made from rubber or other similar waterproof materials. The interface with the ring 802 may be impervious to water and other liquids or fluids. The palm sealing ring 802 may be a separate component that is placed into the palm cover 700 or around the chassis 202, for example into the ridge 1401. In some embodiments, the palm sealing ring 802 may be integrated with one of the components, such as with the palm cover 700, for example with the ridge 1401, or with the chassis 202. In some embodiments, the palm sealing ring 802 may be integrated with one of the components via computer numerical control (CNC) gasket deposition. For instance, a gasket may be laid down and stuck with the component. The palm sealing ring 802 may be made of rigid plastic. which may be overmolded with soft material (such as silicone or rubber). This construction may facilitate with maintaining the shape and structure of the palm sealing ring 802 and better securing it in place.

FIGS. 18A-C show various views of the dorsal side 108 of the prosthetic hand 100 and the dorsal cover 710. FIG. 18A shows a perspective view of the dorsal side 108 of the prosthetic hand 100 with the dorsal cover 710 removed. FIG. 18B shows a perspective view of the exterior surface 710A of the dorsal cover 710. FIG. 18C shows a perspective view of the interior surface 710B of the dorsal cover 710. The dorsal cover 710 may be made of a hard plastic such as a polycarbonate blend, or any other suitable alternative. There may be an over-molded dorsal sealing ring 1402 along a perimeter of the dorsal opening between the interior surface 710B of dorsal cover 710 and the palm cover 700 to prevent water from penetrating the waterproof cover. The dorsal sealing ring 1402 may be made from a soft rubber such as a thermoplastic elastomer (TPE) or any other suitable alternative. The interface with the dorsal sealing ring 1402 may be impervious to water and other liquids or fluids. The dorsal cover 710 may be secured with the prosthetic hand 100 via the dorsal latch opening 1404, and corresponding dorsal latch 1406 on the interior surface of the dorsal cover 710. The opening 1404 may be defined by the chassis 202 with a shape complementary to that of the dorsal latch 1406. A clip 1408 may be located within the opening 1404 or on an interior side of the opening 1404. The dorsal latch 1406 may protrude outwardly away from the interior surface 710B and define a slot 1410 that engages with the clip 1408. The clip 1408 may be flexible. The dorsal cover 710 may be removable if needed. In some embodiments, the removal of one prosthetic digit 104 via the filament securement system may allow a user to unclip the dorsal cover 710 via the dorsal latch securement system.

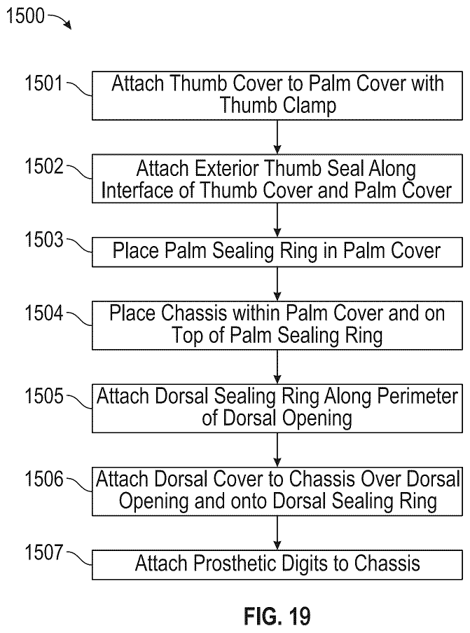

FIG. 19 is a flow chart illustrating an example method 1500 of assembling a cover assembly for a prosthetic hand, such as the cover assembly 800. At step 1501, the thumb cover 702 is attached to the palm cover 700 using the thumb clamp 1004. The method 1500 then moves to step 1502, where the exterior thumb seal 704 is attached along the interface of the thumb cover 702 and the palm cover 700. The method 1500 then moves to step 1503, where the palm sealing ring 802 is placed into the palm cover 700. The method 1500 then moves to step 1504, where the chassis 202 is placed into the palm cover 700 partially on top of the palm sealing ring 802. The method 1500 then moves to step 1505, where the dorsal scaling ring 1402 is secured along a perimeter of the dorsal opening 1412. The method 1500 then moves to step 1506, where the dorsal cover 710 is attached to the dorsal side 108 of the prosthetic hand 100, e.g. using the dorsal latch 1406 and dorsal latch opening 1404 securement system. The method 1500 then moves to step 157, where the prosthetic digits 204 are attached to the prosthetic hand 100, for example, using the filament securement system disclosed herein. Step 157 may include the method 500 shown in FIG. 10.

In some embodiments, steps related to assembling the seal assembly 800 are performed prior to attaching the prosthetic digits 204. For example, steps 1503 and 1504 may be performed prior to step 1507. This allows for simplicity in attaching and removing the prosthetic digits 204. The cover assembly 800 may be assembled prior to or after attaching the prosthetic digits 204, allowing flexibility in the assembly process. Conversely, the prosthetic digits 204 may be removed and replaced from the chassis 202 with the palm cover 700 already on the prosthetic hand 100 and thus without having to disassemble the entire cover assembly 800.

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results, except as otherwise described. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic hand comprising:
a palm portion;
a prosthetic digit and a prosthetic thumb extending from the palm portion;
a chassis located within the palm portion and attached to the prosthetic digit to define an annular groove extending between the prosthetic digit and the chassis;
a flexible filament extending through the groove; and
a cover assembly comprising:
a palm cover extending partially over the palm portion and defining a lateral opening and a dorsal opening;
a thumb cover extending around at least part of the prosthetic thumb and secured to the lateral opening of the palm cover; and
a dorsal cover extending over the dorsal opening.

2. The prosthetic hand of claim 1, wherein the prosthetic digit further comprises a first recess extending annularly about a proximal end of the prosthetic digit and that partially defines the annular groove.

3. The prosthetic hand of claim 2, wherein the chassis further comprises an opening at a distal end of the chassis, the opening having a second recess extending annularly around an inner surface of the opening, wherein the opening is configured to receive therein the proximal end of the prosthetic digit, such that the first recess and the second recess align to define the annular groove between the prosthetic digit and the chassis.

4. The prosthetic hand of claim 1, further comprising a thumb clamp, the thumb clamp adhered to the thumb cover and the lateral opening along a thumb interface, with an exterior thumb seal extending over an exterior of the thumb interface, such that the thumb interface is impermeable to liquid.

5. The prosthetic hand of claim 1, wherein the dorsal cover is rigid and comprises an outer perimeter extending at least partially along a corresponding inner perimeter of the dorsal opening along a dorsal interface, and the dorsal cover is secured to the chassis via a latch, such that the dorsal interface is impermeable to liquid.

6. The prosthetic hand of claim 1, wherein the thumb cover is flexible and comprises an internal elastane layer, a middle rubberized layer, and a woven fabric external layer.

7. The prosthetic hand of claim 1, further comprising four of the prosthetic digits and four of the filaments, with each filament extending through a respective groove defined by the respective prosthetic digit and the chassis.

8. A prosthetic hand comprising:
a prosthetic digit comprising a first recess extending annularly about an outer surface of a proximal end of the prosthetic digit;
a chassis carried by a palm portion of the prosthetic hand, the chassis having a distal end comprising an opening having a second recess extending annularly around an inner surface of the opening, wherein the opening is configured to receive therein the proximal end of the prosthetic digit such that the first recess and the second recess align to define a groove between the prosthetic digit and the chassis; and
a flexible filament extending through the groove and thereby securing the prosthetic digit to the chassis.

9. The prosthetic hand of claim 8, wherein the chassis further comprises an access opening leading to the annular groove, the access opening configured to receive a distal end of the filament therein for insertion of the filament into the groove.

10. The prosthetic hand of claim 9, further comprising a cover extending around the palm portion, and wherein the cover extends over the access opening.

11. The prosthetic hand of claim 9, wherein the access opening is located on a dorsal side of the chassis.

12. The prosthetic hand of claim 8, wherein the first recess and the second recess each have cross sections with a long side and two short sides perpendicular to the long side such that, with the first recess and the second recess aligned, the groove defines a four-sided cross section.

13. The prosthetic hand of claim 8, wherein the groove comprises a proximal end extending to a distal end, and wherein the distal end is open to the proximal end to define a continuous annular pathway.

14. The prosthetic hand of claim 8, wherein the filament comprises a longitudinal core with an outer strand wrapped around the core.

15. A prosthetic hand comprising:
a palm portion having a distal end, a palm side opposite a dorsal side, and a lateral inner side opposite a lateral outer side;
a prosthetic digit attached to and extending from the distal end of the palm portion;
a prosthetic thumb attached to and extending from the lateral inner side of the palm portion; and
a cover assembly comprising:
a polymeric palm cover extending partially around the palm portion and defining a lateral opening on the lateral inner side of the palm portion, a distal opening on the distal end of the palm portion, and a dorsal opening on the dorsal side of the palm portion;
a ridge of the palm cover located proximal of the distal opening and protruding inwardly from and extending at least partially along one or more inner surfaces of the palm cover,
a palm sealing ring extending along the ridge of the palm cover with a chassis compressing the palm sealing ring against the ridge along a palm interface, the palm interface impermeable to liquid;
a flexible thumb cover having a sidewall with a closed cross-section and extending from a proximal thumb opening to a distal thumb opening, the proximal thumb opening attached to the lateral opening of the palm cover and a distal end of the prosthetic thumb extending distally from the distal thumb opening;
a thumb clamp extending along and securing a first perimeter of the lateral opening of the palm cover with a second perimeter of the proximal thumb opening of the thumb cover along a thumb cover interface;
an exterior thumb seal extending over an exterior of the thumb cover interface, wherein the thumb cover interface is impermeable to liquid; and
a rigid dorsal cover having an outer perimeter extending at least partially along a corresponding inner perimeter of the dorsal opening along a dorsal interface, wherein the dorsal interface is impermeable to liquid.

16. The prosthetic hand of claim 15, wherein the exterior thumb seal comprises a deformable grip pad facing outwardly from the palm portion.

17. The prosthetic hand of claim 15, wherein the thumb cover comprises an internal elastane layer, a middle rubberized layer, and a woven fabric external layer.

18. The prosthetic hand of claim 15, further comprising:
a chassis within the palm portion, wherein the prosthetic digit and the chassis define an annular groove; and
a filament located within the groove thereby securing the prosthetic digit to the chassis.

19. The prosthetic hand of claim 15, further comprising:
a dorsal opening defined by the chassis; and
a latch attached to and extending away from an interior of the dorsal cover, the latch secured within the dorsal opening of the chassis.

20. The prosthetic hand of claim 15, further comprising a dorsal sealing ring extending along the inner perimeter of the dorsal opening.

21. A method of attaching a prosthetic digit to a prosthetic hand, the method comprising:
placing a proximal end of a prosthetic digit within an opening of a chassis;
forming a groove along an interface of the prosthetic digit and the chassis; and
inserting a filament via an access opening of the chassis into the groove, thereby securing the prosthetic digit with the chassis.

22. A method of attaching a cover to a prosthetic hand, the method comprising:
inserting a chassis into a palm cover through a distal opening of the palm cover; and
attaching, after the chassis has been inserted into the palm cover, a prosthetic digit to a distal end of the chassis.

23. The method of claim 22, further comprising:
attaching, prior to inserting the chassis into the palm cover, a thumb cover about a prosthetic thumb that is attached to the chassis.

24. The method of claim 22, further comprising attaching a dorsal cover to a dorsal side of the palm cover.
